(19) United States
(12) Patent Application Publication
(10) Pub. No.: US 2026/0033978 A1
(43) Pub. Date: Feb. 5, 2026

(54) FLUID COLLECTION DEVICES, SYSTEMS, AND METHODS SECURING A PROTRUDING PORTION IN POSITION FOR USE

(71) Applicant: PUREWICK CORPORATION, Covington, GA (US)

(72) Inventors: Eric Alan Fallows, Apex, NC (US); Melody Mei Hee Kuroda, Durham, NC (US); Adam Martin, Holly Springs, NC (US); Nicholas Jardine, Holly Springs, NC (US); Florence Stevenson, Baltimore, MD (US); Michael Bielawa, Baltimore, MD (US); Brian Murphy, Baltimore, MD (US); David R. Schiff, Highland Park, NJ (US); Sharon D. West, Norristown, PA (US); Vladlena Belozerova, Philadelphia, PA (US); Breanna Stachowski, Covington, GA (US); Justin Bechstein, Philadelphia, PA (US); Trisha Eckard, Warminster, PA (US)

(21) Appl. No.: 19/356,506

(22) Filed: Oct. 13, 2025

Related U.S. Application Data
(63) Continuation of application No. 17/996,253, now Pat. No. 12,465,514.
(60) Provisional application No. 63/011,760, filed on Apr. 17, 2020.

(51) Int. Cl. A61F 5/443; A61F 5/455

(52) U.S. Cl. CPC A61F 5/443; A61F 5/455

(57) ABSTRACT

Examples relate to systems, devices, and methods utilizing means for securing a protruding portion of a fluid permeable body disposed between a wearer's labia in position during use. The protruding portion is formed from fluid permeable material that allows urine or other fluids to pass therethrough. The fluid permeable body is disposed at least partially within a fluid impermeable barrier to prevent leakage of the fluid. The fluid is removed through a conduit disposed in a chamber within the fluid impermeable barrier.

### CROSS-REFERENCE TO RELATED APPLICATIONS

This application is a continuation of U.S. application Ser. No. 17/996,253 filed Oct. 14, 2022, which is a U.S. Nationalization of International Application No. PCT/US2021/027314 filed on Apr. 14, 2021, which claims priority to U.S. Provisional Patent Application No. 63/011,760 filed on Apr. 17, 2020, the disclosure of each of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, can be used to address some of these circumstances. However, bed pans and urinary catheters have several problems associated therewith. For example, bed pans can be prone to discomfort, pressure ulcers spills, and other hygiene issues. Urinary catheters can be uncomfortable, painful, and can cause urinary tract infections.

Thus, users and manufacturers of fluid collection devices continue to seek new and improved devices, systems, and methods to collect urine.

### SUMMARY

Embodiments disclosed herein relate to fluid collection devices, systems, and methods utilizing means for securing a protruding portion of a fluid permeable body disposed between a wearer's labia in position during use. In an embodiment, a fluid collection device is disclosed. The fluid collection device includes a fluid impermeable barrier at least partially defining a chamber and an opening. The fluid impermeable barrier includes a first end region and a second end region. The fluid collection device includes a fluid permeable body positioned at least partially within the chamber to extend across at least a portion of the opening and configured to allows fluid to move away from the opening. The fluid permeable body includes a protruding portion protruding out of the opening and that is sized and shaped to fit between labia of a wearer. The fluid collection device includes a conduit disposed within the chamber for removing fluids therefrom.

In an embodiment, a fluid collection system is disclosed. The system includes the fluid collection device described above. The system includes a fluid storage container fluidly connected to the fluid collection device via the conduit, the fluid storage container being configured to store fluids therein. The system includes a vacuum source fluidly connected to the fluid storage container, the vacuum source being configured to provide vacuum force into the chamber via the fluid storage container and the conduit.

In an embodiment, a method to collect fluid is disclosed. The method includes positioning the fluid collection device disclosed above adjacent to a urethra of a wearer effective to position the protruding portion between the labia of the wearer. The method includes receiving fluid from the urethra into the fluid collection device. The method includes removing the fluid from the fluid collection device via the conduit.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the invention, wherein identical reference numerals refer to identical or similar elements or features in different views or embodiments shown in the drawings.

FIG. 1 is a schematic diagram of a fluid collection device, according to an embodiment.

FIG. 2 is an isometric view of a fluid collection device, according to an embodiment.

FIG. 3 is a cross sectional view of the fluid collection device of FIG. 2 taken along the plan A-A, according to an embodiment.

FIG. 4 is a cross-sectional view of a wearer having the fluid collection device of FIGS. 2 and 3 positioned thereon during use, according to an embodiment.

FIG. 5 depicts the fluid collection device of FIGS. 2 and 3 positioned on the wearer for use, according to an embodiment.

FIG. 6 is an isometric view of a fluid collection device, according to an embodiment.

FIG. 7 is an isometric view of a fluid collection device, according to an embodiment.

FIG. 8 is an isometric view of a fluid collection device, according to an embodiment.

FIG. 9 is an isometric view of a fluid collection device, according to an embodiment.

FIG. 10 is an isometric view of the fluid collection device of FIG. 9 being positioned on the wearer, according to an embodiment

FIG. 11 is an isometric view of a fluid collection device, according to an embodiment

FIG. 12 is an isometric view of a fluid collection device, according to an embodiment.

FIG. 13 is an isometric view of the fluid collection device of FIG. 12 positioned on a wearer, according to an embodiment.

FIG. 14 is side view of the fluid collection device of FIG. 12 positioned on a wearer, according to an embodiment.

FIGS. 15 and 16 are isometric views of the fluid collection device of FIG. 12 during and after positioning on a wearer, according to embodiments.

FIG. 17 is an isometric view of a fluid collection device viewed from the wearer-facing side, according to an embodiment.

FIG. 18 is an isometric view of the fluid collection device disposed on a wearer, according to an embodiment.

FIG. 19 is an isometric view of a fluid collection device, according to an embodiment.

FIG. 20 is an isometric view of the foldable body in different folded configurations, according to an embodiment.

FIG. 21 is an isometric view of the fluid collection device of FIG. 19 disposed on a wearer, according to an embodiment.

FIG. 22 is an isometric view of a fluid collection device, according to an embodiment.

FIGS. 23 and 24 are isometric views of the fluid collection device of FIG. 22 disposed on a wearer, according to embodiments.

FIG. 25 is a block diagram of a system for collecting fluid, according to an embodiment

FIG. 26 is a flow diagram of a method for collecting fluid, according to an embodiment.

### DETAILED DESCRIPTION

Embodiments disclosed herein include fluid collection devices and systems utilizing portions sized and shaped to be inserted between labia for securement and urine capture, as well as methods of using the fluid collection devices and systems. The embodiments utilize means for securing a protruding portion of a fluid permeable body disposed between a wearer's labia in position during use. Example fluid collection devices include a fluid impermeable barrier defining an interior chamber for collecting fluids. The fluid collection devices include a fluid permeable body at least partially disposed within the fluid impermeable barrier and exposed through an opening therein. The fluid collection devices include a fluid permeable body having at least one permeable (e.g., porous) material for collecting and passing fluids therethrough, such as a wicking material (e.g., foam or spun plastic fibers). The fluid permeable body includes a protruding portion sized and shaped to protrude out of the opening of the fluid impermeable barrier. The protruding portion is sized and shaped to fit, and be retained, between the labia of a female wearer. The fluid collection devices include a means for retaining the protruding portion between the labia of the wearer. Accordingly, the devices, systems, and methods disclosed herein provide for positioning and retaining of the protruding portion of the fluid permeable body over the urethra within the labia of the wearer.

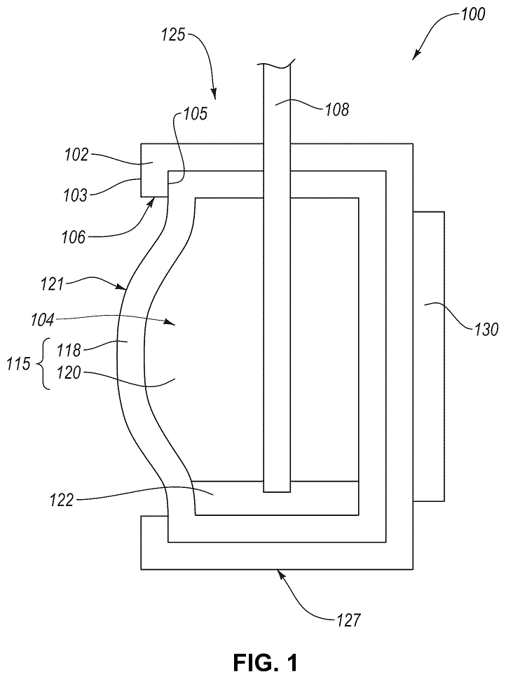

FIG. 1 is a block diagram of a fluid collection device 100, according to an embodiment. The fluid collection device 100 is sized, shaped, and composed to collect urine from the urethra of a wearer. The fluid collection device 100 includes a fluid impermeable barrier 102 defining a chamber 104 therein, a fluid permeable body 115 disposed in the chamber 104 (e.g., interior chamber) and having a protruding portion 121 protruding therefrom, a conduit 108 disposed in the 20) chamber 104, and one or more means 130 for retaining the protruding portion 121 between the labia of the wearer. The protruding portion 121 of the fluid permeable body 115 may be exposed to the external environment via an opening 106 in the fluid impermeable barrier 102. During use, the fluid collection device 100 may be positioned over the urethra of the wearer with the protruding portion 121 disposed between the labia of the wearer. Accordingly, urine may be received in the protruding portion 121 and moved into the chamber 104 via the opening 106. The urine may be removed from the fluid collection device 100 via the conduit 108 disposed within the chamber 104.

The fluid impermeable barrier 102 at least partially defines the chamber 104 and opening 106. For example, the inner surface(s) 103 of the fluid impermeable barrier 102 at least partially defines the chamber 104 within the fluid collection device 100. The fluid impermeable barrier 102 at least temporarily retains the fluid(s) in the chamber 104. The fluid impermeable barrier 102 may be formed of any suitable fluid impermeable material(s), such as a fluid impermeable polymer (e.g., silicone, polypropylene, polyethylene, polyethylene terephthalate, thermoplastic elastomer(s), a polycarbonate, etc.), a metal film, natural rubber, another suitable material, or combinations thereof. As such, the fluid impermeable barrier 102 substantially prevents the fluid(s) from passing therethrough. In an example, the fluid impermeable barrier 102 may be air permeable and liquid impermeable. In such an example, the fluid impermeable barrier 102 may be formed of a hydrophobic material that defines a plurality of pores that are air permeable but not liquid permeable. In an example, one or more portions of at least an outer surface of the fluid impermeable barrier 102 may be formed from a soft and/or smooth material, thereby reducing chaffing.

In some examples, the fluid impermeable barrier 102 may be tubular (ignoring the opening), such as substantially cylindrical, oblong, prismatic, flattened tube, or any other extruded shape. The fluid impermeable barrier 102 may be sized to fit between the legs of a wearer. During use, an outer surface 105 of the fluid impermeable barrier 102 may at least partially contact the wearer, such as the thighs of the wearer.

The opening 106 provides an ingress route for fluids to enter the chamber 104. The opening 106 may be defined by the fluid impermeable barrier 102, such as by an inner edge of the fluid impermeable barrier 102. For example, the opening 106 is formed in and extends through the fluid impermeable barrier 102, from the outer surface 105 to the inner surface 103, thereby enabling fluid(s) to enter the chamber 104 from outside of the fluid collection device 100. The opening 106 may be longitudinally extending along a longitudinal axis of the fluid collection device 100. At least a portion of porous material(s) of the fluid permeable body 115 disposed in the chamber 104 may be exposed through the opening 106 to allow fluids to move inwardly into the chamber 104, such as via one or more of permeation, suction, gravity, or wicking. For example, the fluid permeable body 115 includes the protruding portion 121 which extends through the opening 106. The protruding portion 121 receives fluid which is moved through the fluid permeable body 115 into the chamber 104.

The protruding portion 121 of the fluid collection device 100 may be positioned proximate to the urethra and urine may enter the chamber 104 via the opening 106. When in use, the opening 106 may be elongated, longitudinally extending from a first location below the urethra to a second location above the urethra (e.g., at or near the top of the vaginal opening or the pubic region). The opening 106 in the fluid impermeable barrier 102 may exhibit a length that is measured along the longitudinal axis of the fluid collection device 100 that may be at least about 10% of the length of the fluid collection device 100, such as about 25% to about 50%, about 40% to about 60%, about 50% to about 75%, about 65% to about 85%, or about 75% to about 95% of the length of the fluid collection device 100.

The opening 106 in the fluid impermeable barrier 102 may exhibit a width that is measured transverse to the longitudinal axis of the fluid collection device 100 and may be at least about 10% of the circumference of the fluid collection device 100, such as about 25% to about 50%, about 40% to about 60%, about 50% to about 75%, about 65% to about 85%, or about 75% to about 100% of the circumference of the fluid collection device 100. The opening 106 may exhibit a width that is greater than 50% of the circumference of the fluid collection device 100 because the vacuum (e.g., suction) through the conduit 108 pulls the fluid through the fluid permeable body 115 and into the conduit 108. The opening 106 may be longitudinally oriented (e.g., having a major axis parallel to the longitudinal axis of the device 100) or laterally oriented (e.g., having a major axis perpendicular to the longitudinal axis of the device 100).

The fluid collection device 100 includes the fluid permeable body 115 disposed in the chamber 104. The fluid permeable body 115 may extend across at least a portion (e.g., all) of the opening 106. At least a portion of the fluid permeable body 115 may be exposed to an environment outside of the chamber 104 through the opening 106. The fluid permeable body 115 may wick any fluid away from the opening 106, thereby preventing the fluid from escaping the chamber 104.

The fluid permeable body 115 may include one or more of a fluid permeable membrane 118 or a fluid permeable support 120. In some examples, the fluid permeable support 120 and the fluid permeable membrane 118 may be made of solely porous material(s), such as a wicking material. At least a portion of the porous material of the fluid permeable body 115 may be a wicking material configured to wick, draw, and/or allow transport any of the bodily fluids away from the opening 106, thereby preventing bodily fluids from escaping the chamber 104. The porous material may not include absorption of the bodily fluids into at least a portion of the porous material. Put another way, substantially no absorption or solubility of the bodily fluids into the porous material may take place after the porous material is exposed to the bodily fluids. While no absorption is desired, the term “substantially no absorption” may allow for nominal amounts of absorption and/or solubility of the bodily fluids into the porous material (e.g., absorbency), such as about 30 wt % of the dry weight of the porous material, about 20%, about 10%, about 7 wt %, about 5 wt %, about 3 wt %, about 2 wt %, about 1 wt %, or about 0.5 wt % of the dry weight of the porous material. In some examples, the porous material may include at least one absorbent or adsorbent material.

The fluid permeable membrane 118 may include any porous material or a material that may wick the fluid. For example, the fluid permeable membrane 118 may include fabric, such as a gauze (e.g., a silk, linen, or cotton gauze), another soft fabric, or another smooth fabric. The fluid permeable membrane 118 may include spun plastic fibers (e.g., nylon), such as a spun plastic mat or bed. Forming the fluid permeable membrane 118 from gauze, soft fabric, and/or smooth fabric may reduce chaffing caused by alternative materials.

The fluid permeable membrane 118 is at least partially disposed in the chamber 104. The fluid permeable membrane 118 may extend across or out of at least a portion (e.g., all) of the opening 106. The fluid permeable membrane 118 may wick fluid inwardly away from the opening 106, thereby preventing fluid from escaping the chamber 104.

The fluid permeable body 115 of the fluid collection device 100 may include the fluid permeable support 120 disposed in the chamber 104. The fluid permeable support 120 is composed to support the fluid permeable membrane 118 since the fluid permeable membrane 118 may be formed from a foldable, flimsy, or otherwise easily deformable material. For example, the fluid permeable support 120 may be positioned such that the fluid permeable membrane 118 is disposed between the fluid permeable support 120 and the fluid impermeable barrier 102. As such, the fluid permeable support 120 may support and maintain the position of the fluid permeable membrane 118 thereon. The fluid permeable support 120 may include any material that may wick the fluid, such as any of the fluid permeable membrane materials disclosed herein. For example, the fluid permeable support 120 may be formed from any fluid porous material that is less deformable than the fluid permeable membrane 118, such as any of the materials disclosed herein for the fluid permeable membrane 118, in a more dense or rigid form. In some examples, the fluid permeable support 120 may include a porous polymer (e.g., nylon, polyester, polyurethane, polyethylene, polypropylene, etc.) structure, an open cell foam, or spun plastic fibers (e.g., spun nylon fibers). In some examples, the fluid permeable membrane 118 may include gauze and the fluid permeable support may include spun nylon fibers. In some examples, the fluid permeable support 120 may be formed from fabric, felt, gauze, or combinations thereof. In some examples, the fluid permeable support 120 may be formed from a natural material, such as cotton, wool, silk, or combinations thereof. In such examples, the material may have a coating to prevent or limit absorption of fluid into the material, such as a water repellent coating. In some examples, the fluid permeable support 120 may be omitted from the fluid collection device 100. In some examples, the fluid permeable membrane 118 may be optional. For example, the fluid permeable body 115 may include only the fluid permeable support 120.

The fluid permeable support 120 may have a greater permeability or a greater ability to wick fluids than the fluid permeable membrane 118, such as to move the fluid inwardly from the outer surface of the fluid collection device 100. In some examples, the permeability or the wicking ability of the fluid permeable support 120 and the fluid permeable membrane 118 may be substantially the same.

The fluid permeable body 115 includes the protruding portion 121. The protruding portion 121 extends out of the chamber 104 through the opening 106. The protruding portion 121 may be a longitudinally extending ridge, fin, mound, or the like that extends out of the opening 106. The protruding portion 121 may include one or more of the fluid permeable membrane 118 or the fluid permeable support 120. For example, the protruding portion 121 may include only the fluid permeable membrane 118 such as a thicker portion of the fluid permeable membrane 118 disposed in and extending from the opening 106. The protruding portion 121 may have a substantially arcuate shape when viewed transverse to the longitudinal axis of the fluid collection device.

The protruding portion 121 may extend longitudinally along at least a portion of the opening 106, such as along at least 20% of the length of the opening 106. For example, the protruding portion 121 may have a length that is at least about 1 cm, such as 1 cm to 15 cm, 2 cm to 5 cm, 5 cm to 10 cm, 10 cm to 15 cm, or less than 20 cm. The protruding portion 121 may have a width that is 50% or less than the length of the protruding portion 121, such as 1% to 50%, 5% to 25%, or 25% to 50% of the length of the protruding portion 121. The height (above the fluid impermeable barrier 102 at the opening 106) of the protruding portion 121 may be at least 0.5 cm, such as 0.5 cm to 5 cm, 0.5 cm to 2 cm, 2 cm to 5 cm, or less than 5 cm.

The fluid permeable membrane 118 and the fluid permeable support 120 may at least substantially completely fill the portions of the chamber 104 that are not occupied by the conduit 108. In another example, the fluid permeable membrane 118 and the fluid permeable support 120 may not substantially completely fill the portions of the chamber 104 that are not occupied by the conduit 108. In such an example, the fluid collection device 100 includes a reservoir 122 in the chamber 104.

The conduit 108 extends into the chamber 104. The conduit 108 may be at least partially disposed in the chamber 104. The conduit 108 (e.g., a drainage tube) includes an inlet and outlet positioned downstream from the inlet. The conduit 108 may extend into the chamber 104 to any point therein. For example, the conduit 108 may be inserted into the chamber 104 at a first end region 125 of the fluid collection device 100 and extend therethrough into the first end region 125 or to the second end region 127. The conduit 108 may extend into the fluid impermeable barrier 102 from the first end region 125 through to the reservoir 122 such that the inlet of the conduit 108 is in fluid communication with the reservoir 122. The fluid collected in the reservoir 122 may be removed from the chamber 104 via the conduit 108.

In some examples, the conduit 108 may enter the chamber 104 in the second end region 127 and the inlet of the conduit 108 may be disposed in the second end region 127. The reservoir 122 may be disposed in the second end region 127 in any of the embodiments disclosed herein. The inlet may be spaced from inner surface 103 of the fluid permeable support 120 in the first end region. In some examples, the inlet may be disposed at the end of the fluid permeable support 120 in the first end region 125, such as flush with the end of the fluid permeable support 120. In some examples, the inlet may be disposed within the fluid permeable support 120 such between the first end region 125 and the second end region 127.

The conduit 108 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some examples, the conduit 108 may include silicon or latex. In some examples, the conduit 108 may include one or more portions that are resilient, such as to by having one or more of a diameter or wall thickness that allows the conduit 108 to be flexible. The conduit 108 may be at least partially transparent. In some examples, one or more portions of the conduit 108 may be frosted or opaque (e.g., black) to obscure visibility of the fluid(s) therein.

The fluid impermeable barrier 102, the fluid permeable membrane 118 and the fluid permeable support 120 may be sized and shaped to have the conduit 108 at least partially disposed in the chamber 104. For example, at least one of the fluid permeable membrane 118 and the fluid permeable support 120 may be configured to form a space that accommodates the conduit 108. The fluid impermeable barrier 102 may define an aperture sized to receive the conduit 108. The conduit 108 may be disposed in the chamber 104 via the aperture. The aperture may be sized and shaped to form an at least substantially fluid tight seal against the conduit 108, thereby substantially preventing the fluid(s) from escaping the chamber 104.

The fluid permeable body 115 may not substantially completely fill the portions of the chamber 104 that are not occupied by the conduit 108. The fluid collection device 100 may include the reservoir 122 therein. The reservoir 122 is a substantially unoccupied portion of the chamber 104. The reservoir 122 may be defined between the fluid impermeable barrier 102 and the fluid permeable body 115 (e.g., one or both of the fluid permeable membrane 118 and the fluid permeable support 120). The fluid(s) emitted by the wearer may be drawn into the chamber 104 by the fluid permeable body 115 and may flow through the fluid permeable membrane 118 and/or fluid permeable support 120 to the reservoir 122. The fluid impermeable barrier 102 may retain the fluid(s) in the reservoir 122. The reservoir 122 may be located in a portion of the fluid collection device expected to be positioned in a gravimetrically low point of the fluid collection device when worn by a user. In such examples, the location of the inlet of the conduit 108 and the reservoir 122 at the gravimetrically low point of the fluid collection device allows the fluids collected in the chamber 104 to drain into the reservoir 122 when the device is positioned on the wearer. Locating the inlet of the conduit 108 at or near a location expected to be the gravimetrically low point of the chamber 104 when worn by a user enables the conduit 108 to receive more of the fluid(s) than if inlet was located elsewhere and reduce the likelihood of pooling (e.g., pooling of the fluid(s) may cause microbe growth and foul odors). For instance, the fluid(s) in the fluid permeable body 115 may flow in any direction due to capillary forces. However, the fluid(s) may exhibit a preference to flow in the direction of gravity, especially when at least a portion of the fluid permeable body 115 is saturated with the fluid(s). Accordingly, one or more of the inlet or the reservoir 122 may be located in the first end region. While depicted in the first end region, the reservoir 122 may be located in any portion of the chamber 104 such as the second end region 127 in a medial region between the first and second regions. In such examples, the conduit 108 may extend into the reservoir 122, such as through one or more of the fluid permeable body 115 or fluid impermeable barrier 102 in the second end region 127 or the medial region.

Other embodiments of fluid impermeable barriers, fluid permeable membranes, fluid permeable supports, cavities, conduits and their shapes and configurations are disclosed in U.S. patent application Ser. No. 15/612,325 filed on Jun. 2, 2017; U.S. patent application Ser. No. 15/260,103 filed on Sep. 8, 2016; and U.S. Pat. No. 10,226,376 filed on Jun. 1, 2017, the disclosure of each of which is incorporated herein, in its entirety, by this reference.

The fluid collection device 100 includes one or more means 130 for retaining the protruding portion between the labia of the wearer. The one or more means 130 for retaining the protruding portion between the labia of the wearer may be integrated into the construction of one or more of the fluid impermeable barrier 102 or the fluid permeable body 115. The one or more means 130 for retaining the protruding portion between the labia of the wearer may be additional components, such as flaps, frames, straps, bands, adhesives, or the like attached to the fluid collection device 100. Various examples of the one or more means 130 for retaining the protruding portion between the labia of the wearer are discussed in more detail below.

During use, the protruding portion 121 the fluid collection device 100 may be positioned over the urethra and between the labia of the wearer. Urine may be collected in the fluid collection device 100 and removed from the wearer to the fluid storage container, such as via gravity or vacuum force. At least a portion of bodily waste (e.g., liquid or semi-liquid stool) may be collected in the bodily waste collection apparatus and removed to the at least one fluid storage container, such as via gravity or vacuum force.

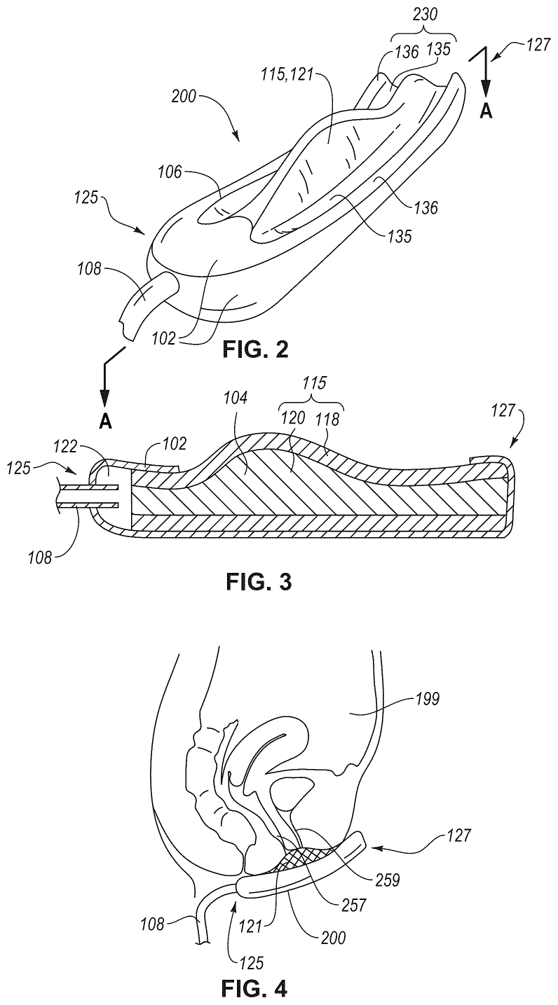

FIG. 2 is an isometric view of a fluid collection device 200, according to an embodiment. FIG. 3 is a cross sectional view of the fluid collection device 200 of FIG. 2 taken along the plan A-A, according to an embodiment. The fluid collection device 200 may be similar or identical to the fluid collection device 100, in one or more aspects. The fluid collection device 200 includes the fluid impermeable barrier 102 having an opening 106 around an outer portion of a wearer-facing side thereof. The fluid impermeable barrier 102 defines the chamber 104 therein. The fluid collection device 200 includes the fluid permeable body 115 that transports urine from the wearer's urethra into the chamber 104 and eventually into the conduit 108, thereby preventing the device from retaining urine against the wearer's body. The fluid permeable body 115 includes the protruding portion 121 which protrudes from chamber 104 via the opening 106. As shown, the protruding portion 121 may be a longitudinally extending portion of the fluid permeable body 115 that is sized and shaped to fit between the labia of the wearer. The protruding portion 121 may have any of the dimensions for the protruding portion disclosed herein, such as length, width, height, or relationships therebetween. The protruding portion 121 extends out of and away from the chamber 104 via the opening 106. The protruding portion 121 may include one or both of the fluid permeable support 120 or the fluid permeable membrane 118 (e.g., soft wicking material). The protruding portion 121 may be convex, such as having a first radius of curvature extending longitudinally along the longitudinal axis of the fluid collection device 100 and a second radius of curvature transverse to the first radius of curvature. The second radius of curvature may be greater than the first radius of curvature, such as at least 3 times larger, at least 10 times larger, or 3 times to 30 times larger than the second radius of curvature. The protruding portion 121 may have an apex of the first radius of curvature at a midpoint along the longitudinal axis of the fluid collection device 100. The protruding portion 121 may have an apex of the first radius of curvature below a midpoint of the longitudinal axis of the fluid collection device 100 (e.g., closer to the first end region 125 than the second end region 127). The protruding portion 121 may have an apex of the first radius of curvature above a midpoint of the longitudinal axis of the fluid collection device 100 (e.g., closer to the second end region 127 than the first end region 125).

The fluid collection device 100 may exhibit a boat shape with a generally flat wearer-facing (e.g., front) surface. While not planar or perfectly flat, the front surface may be less than cylindrical, such as rounded in one or more portions thereof.

The fluid collection device 200 includes the means 230 for retaining the protruding portion 121 between the labia of the wearer. The means 230 for retaining the protruding portion 121 between the labia of the wearer may include geometry on at least a portion of the wearer-facing surface that is sized and shaped to accommodate the labia therein. For example and as shown in FIG. 3, at least a portion of one or more of the fluid impermeable barrier 102 and the fluid permeable body 115 may be shaped to accommodate the labia therein. For example, means 230 for retaining the protruding portion 121 between the labia of the wearer may include valleys 135 defined between the protruding portion 121 and a raised edge 136. The valleys 135 may be longitudinally extending in parallel to the protruding portion 121. The valleys 135 are sized and shaped to contour and fit over the labia. The valleys 135 may contain holes or material to capture overflow and guide urine to the chamber for collection and transport. In some examples, the valleys 135 are formed in the fluid permeable body 115 which is exposed through the opening 106 defined at least in part by the raised edge 136. In examples, the opening 106 extends to the second end region 127 of the fluid collection device 200, but not to the first end region 125. In such examples, the end of the fluid collection device 200 in the second end region 127 may be open (e.g., the fluid permeable body 115 is exposed on the end) or may be capped by the fluid impermeable barrier 102. In some examples, the valleys 135, raised edge 136, opening 106, and protruding portion 121 may be disposed in the medial portion the fluid collection device 200.

The protruding portion 121 is sized and shaped to be placed between a wearer's labia and contact urethral opening or tube to promote urine capture by the fluid permeable body 115. FIG. 4 is a cross-sectional view of a wearer 199 having the fluid collection device 200 of FIGS. 2 and 3 positioned thereon during use. As shown, the protruding portion 121 of the fluid collection device 200 may be positioned near the urethra 257 and the wearer's vagina 259. Accordingly, fluids from the urethra 257 and vagina 259 may be collected by the protruding portion 121 and moved into the chamber of the fluid collection device 200. As shown, the first end region 125 may be disposed nearer the rectum of the wearer and the second end region 127 may be disposed nearer the pubic region of the wearer. Accordingly and as shown in FIG. 3, the first end region 125 may include the reservoir 122 and the inlet of the conduit 108 therein. Of course, the reservoir 122 and inlet of the conduit 108 may be disposed in the second end region 127 in one or more embodiments, such as when the position of the device is reversed. In such examples, the conduit 108 may enter the fluid collection device 200 in the first end region 125 and extend through the fluid permeable body 115 (e.g., fluid permeable support 120) to the second end region 127 where the reservoir 122 is disposed.

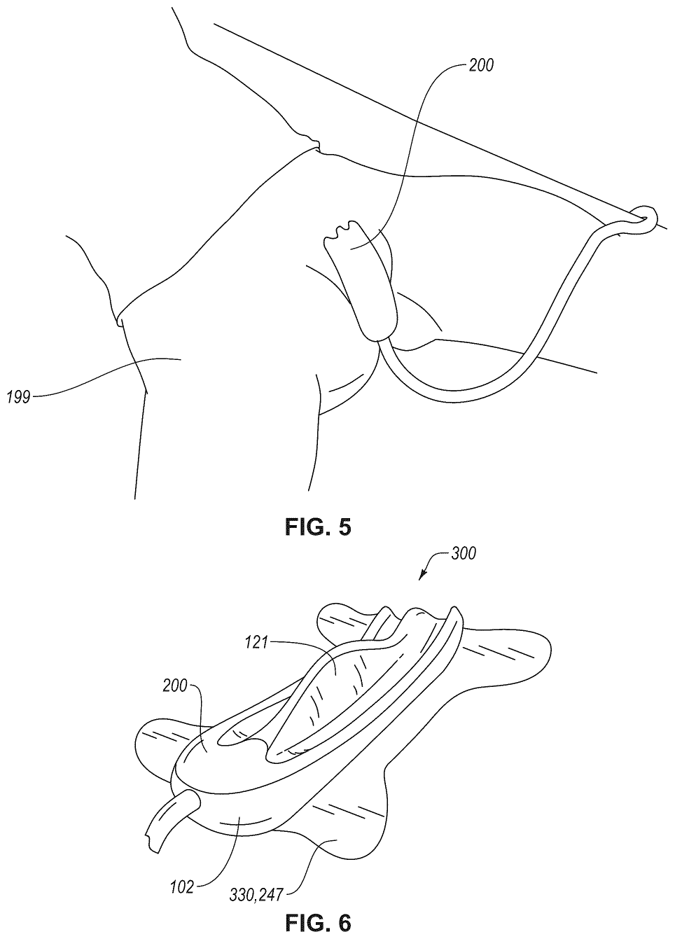

FIG. 5 depicts the fluid collection device 200 of FIGS. 2 and 3 positioned on the wearer 199 for use, according to an embodiment. The fluid collection device 200 can be secured via the labia only, such as via inward pressure applied by the labia, fit or pressure on the labia supplied by the means 230 for retaining the protruding portion 121 between the labia of the wearer, or both. Accordingly, the fluid collection device 200 may only be passively retained in place.

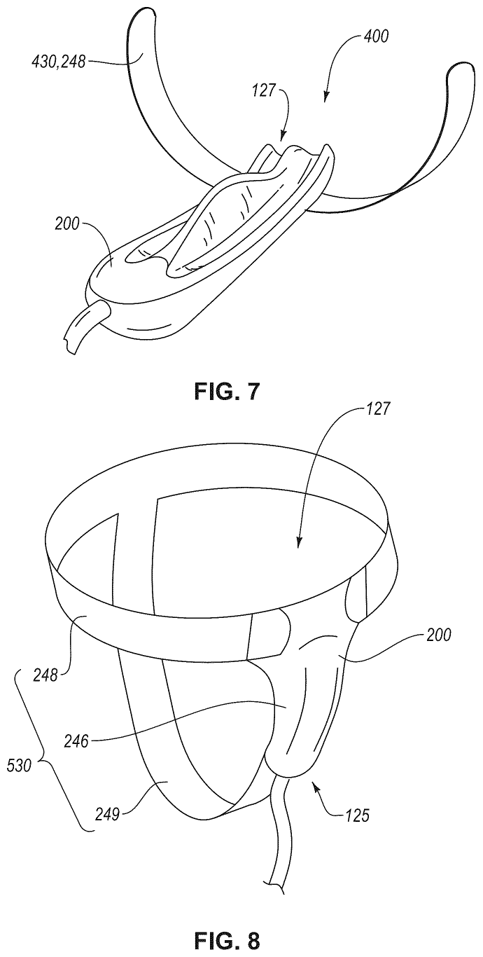

In some examples, other means (e.g., securement structures and approaches) may be used to retain the protruding portion 121 between the labia of the wearer, such as adhesive tabs, use of positioning in the gluteal cleft, straps around the waist and/or buttocks area. For example, adhesive flaps may be used to added to further secure to legs, pelvis, or buttocks. FIG. 6 is an isometric view of a fluid collection device 300, according to an embodiment. The fluid collection device 300 may include the fluid collection device 200 having one or more adhesive flaps 247 thereon. The one or more adhesive flaps 247 provide means 330 for retaining the protruding portion 121 between the labia of the wearer. Adhesive flaps 247 may be disposed on and extend from the fluid impermeable barrier 102. Alternatively, the adhesive flaps 247 may be sized and shaped to fit over the fluid collection device 200, such as extending from the buttocks to the pubic region of the wearer. The adhesive on the adhesive flaps 247 may be disposed on the wearer-facing side (e.g., front) of the flaps or on the back of the flaps. The adhesive may include a hydrogel or any other skin safe adhesive. The adhesive flaps 247 may include any of the materials for the fluid impermeable barrier 102, a fabric, a polymer, or the like. The adhesive flaps 247 may be attached to the fluid impermeable barrier 102 via an adhesive, fasteners, integral formation with the fluid impermeable barrier, welding, or the like.

Adhesive flaps 247 may extend longitudinally or laterally from (with respect to the longitudinal axis) the fluid impermeable barrier. The adhesive flaps 247 are sized and positioned on the fluid impermeable barrier 102 to secure to the legs, pelvis, or buttocks of the wearer. For example, the adhesive flaps 247 may extend a distance to allow the adhesive thereon to bond to one or more of the wearer's hips, buttocks, pelvis, pubic region, or sacrum. Accordingly, the means 330 for retaining the protruding portion 121 between the labia of the wearer provided by the adhesive flaps 247 retains the protruding portion between the labia of the wearer and in contact with the urethral opening of the wearer during use.

The means for retaining the protruding portion 121 between the labia of the wearer may include one or more straps sized and configured to wrap around one or more anatomical features of the wearer, such as one or more waist or leg straps. FIG. 7 is an isometric view of a fluid collection device 400, according to an embodiment. The fluid collection device 400 may include the fluid collection device 200 having a waistband 248 thereon. Any of the fluid collection devices disclosed herein may be used. The waistband 248 may include waist straps extending from the fluid permeable body. The waistband 248 provides the means 430 for retaining the protruding portion 121 between the labia of the wearer.

The waistband 248 may include any of the materials for the fluid impermeable barrier 102, a fabric, a polymer, or the like. The waistband 248 may be attached to the fluid collection device 200 at the second end region 127 thereof, such as via an adhesive, fastener(s), welding, or integral formation with the fluid impermeable barrier. The waistband 248 may have a length selected to extend an entire distance around a wearer's waist or less than the circumference of the wearer's waist. In the former case, the waistband 248 may include attachments on the ends thereof to connect to secure the waistband around the waist of the wearer. In the latter case, the waistband 248 may include attachments on the ends thereof to attach to the wearer (e.g., hips of the wearers) or the wearer's clothes. As shown, the waste straps of the waistband 248 extend from the second end region 127 of the fluid collection device 200 to at least partially wrap around the waist of the wearer to retain the protruding portion between the labia of the wearer.

FIG. 8 is an isometric view of a fluid collection device 500, according to an embodiment. The fluid collection device 500 includes the fluid collection device 200 (or any other fluid collection device disclosed herein) and means 530 for retaining the protruding portion between the labia of the wearer. The means 530 for retaining the protruding portion 121 between the labia of the wearer includes one or more straps, such as the waistband 248 and a strap 249. The means 530 may include the additional strap 249 extending under the perineal region of the wearer for better retention of the device. The straps may include fabric, gauze, adhesive, hook and loop fasteners, polymer material (e.g., the same material as the fluid impermeable barrier 102), or other components.

As shown in FIG. 8, the straps may be formed into a garment, such as a belt or undergarment with an optional cloth portion 246 positioned to the cover and retain the fluid collection device 200 therein when the means 530 is used. For example, the one or more straps of the means 530 include waistband 248 having the cloth portion 246 in a front region thereof and the strap 249 extending from the cloth portion 246 to a back of the waistband 248 to form an undergarment. The cloth portion 248 may be made of cloth, polymer, or the like.

In some examples, the cloth portion 246 may not be present. In such examples, the waistband 248 may be attached directly to the fluid collection device 200 in the second end region 127 and the strap 249 may be directly attached to the first end region 125 of the fluid collection device 200 and the back of the waistband 248 to form an undergarment including the fluid collection device 200 therein. In either configuration, the means 530 may at least partially wrap around the waist of the wearer and the perineal region of the wearer to retain the protruding portion of the fluid collection device in position on the wearer.

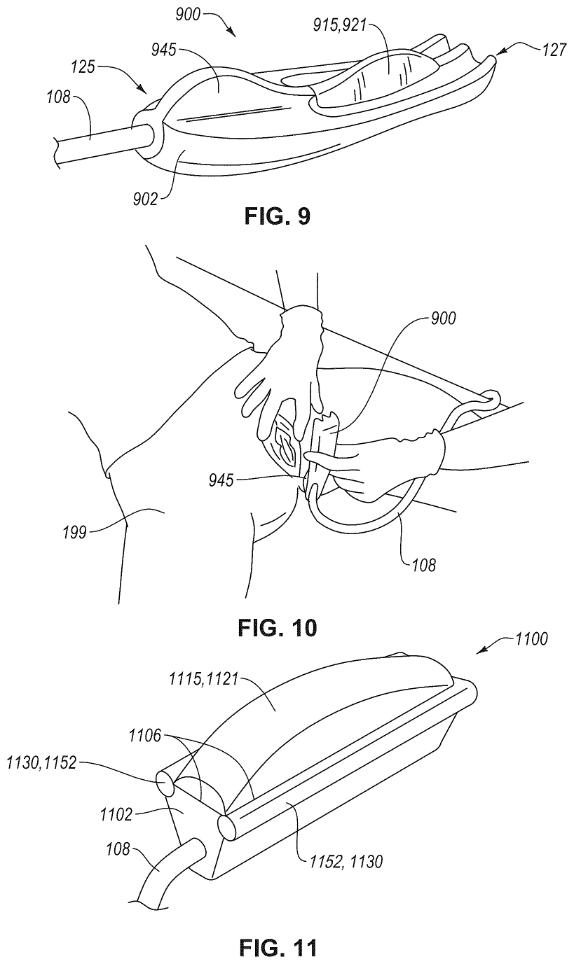

In some examples, the device may include an additional protrusion for securing the device in position. FIG. 9 is an isometric view of a fluid collection device 900, according to an embodiment. The fluid collection device 900 may be similar or identical to the fluid collection device 200 in one or more aspects. For example, the fluid collection device 900 includes a fluid impermeable barrier 902 which is similar or identical to the fluid impermeable barrier 102 in one or more aspects. The fluid collection device 900 includes a fluid permeable body 915 which is similar or identical to the fluid permeable body 115 in one or more aspects. The fluid permeable body 915 includes the protruding portion 921, which is similar or identical to the protruding portion 121 in one or more aspects. As shown, the fluid collection device 900 includes the means 930 for retaining the protruding portion between the labia of the wearer. The means 930 includes an additional protrusion 945 for inserting between the wearer's buttocks to position and retain the protruding portion 921 between the labia. For example, the additional protrusion 945 may be a longitudinally extending protrusion along the same plane as the protruding portion 921. The additional protrusion 945 may be formed in one or both of the fluid impermeable barrier 902 or the fluid permeable body 915. The additional protrusion 945 may extend from the first end region 125 of the fluid impermeable barrier 902 on a wearer-facing side thereof. The additional protrusion 945 may extend from the first end region 125 toward the medial region of the fluid collection device 900. The additional protrusion 945 may have any of the dimensions of the protruding portion 121 disclosed herein. The additional protrusion 945 may have a substantially arcuate shape when viewed transverse to the longitudinal axis of the fluid collection device 900.

The additional protrusion 945 is sized and shaped to fit between the buttocks of the wearer to secure the device in position with the protruding portion 921 being simultaneously positioned between the labia. FIG. 10 is an isometric view of the fluid collection device 900 of FIG. 9 being positioned on the wearer 199, according to an embodiment. The additional protrusion 945 may be inserted between buttocks of the wearer 199. For example, as shown in FIG. 10, the labia may be spread open and the additional protrusion may be placed between the buttocks. The fluid collection device 900 may then be pivoted about the additional protrusion 945 until the protruding portion 921 is positioned between the labia near the urethra. The conduit may be coupled to the fluid collection device 900 at the gravimetrically low portion of the device, such as nearest the perineal region.

In some examples, the fluid collection device may be sized, shaped, and composed to comply to inward pressure (e.g., pinched) to be placed into position between the labia of the wearer. Such device may include means for retaining the protruding portion between the labia. FIG. 11 is an isometric view of a fluid collection device 1100, according to an embodiment. The fluid collection device 1100 includes to fluid impermeable barrier 1102, the fluid permeable body 1115, and the conduit 108. The fluid impermeable barrier 1102 may be similar or identical to any of the fluid impermeable barriers disclosed herein, in one or more aspects. The fluid impermeable barrier 1102 may be shaped as a trough with an at least partially open wearer-facing (e.g., front or upper) surface. For example, the opening 1106 is disposed at a wearer-facing side of the trough.

The fluid permeable body 1115 may be similar or identical to any of the fluid permeable bodies disclosed herein, in or more or aspects. For example, the fluid permeable body 1115 may include the fluid permeable support, the fluid permeable membrane, and protruding portion 1121. The fluid permeable body 1115 is disposed at least partially within the chamber of the fluid impermeable barrier 1102. The protruding portion 1121 may extend out of and above the opening 1106 (e.g., top of the trough). The protruding portion 1121 may extend farther above the opening 1106 when the fluid collection device is pinched.

The protruding portion 1121 may be placed between the wearer's labia such as to contact the urethral tube or opening to promote urine capture. The fluid permeable membrane (e.g., hydrophilic plastic mesh) may disposed over the top of the fluid permeable support in the trough to wick fluid into the fluid permeable support. The conduit 108 may be disposed in the bottom of the trough to remove the collected urine therefrom. The fluid permeable body 1115 (e.g., support and membrane) may alternatively be curved outward laterally or longitudinally to self-center on the vagina of the wearer. Other shapes may be utilized to provide a contoured fit or provide greater engagement with the anatomy of the wearer.

The fluid impermeable barrier 1102 of the fluid collection device 1100 may be semi-rigid with one or more resilient portions composed to maintain their shape therein. For example, the fluid impermeable barrier 1102 may include one or more spring members 1152 extending along the longitudinal axis of the device, such as along the opening 1106 (e.g., top of the trough). The spring members 1152 may include one or more materials or bodies having a greater resiliency or rigidity than the rest of the fluid impermeable barrier 1102. For example, the spring members 1152 may include a polymer, a metal material, or the like having a greater resiliency or rigidity than the fluid impermeable barrier 1102 or material thereof. The spring members 1152 may be at least partially disposed in the fluid impermeable barrier 1102, such as having the material of the fluid impermeable barrier 1102 thereover. The spring members 1152 may be spiraled bodies of resilient at least semi-rigid material (e.g., lateral bending spring), a rod (e.g., torsion bar spring), or the like, extending longitudinally along the opening 1106. The spring members 1152 are sized, shaped, and composed of material to provide an outward bias on the opening 1106. Accordingly, when the fluid collection device 1100 is pinched for the fluid permeable body 1115 to be placed into position between the labia of the wearer, the spring members 1152 provide an outward force keeping the trough open after the inward force is released (at which point the only inward force on the device is provided by the labia of the wearer). The spring members 1152 then provide an outward force against the labia to maintain position of the protruding portion between the labia. Accordingly, the spring members 1152 provide a means 1130 for retaining the protruding portion between the labia of the wearer.

To place the device, a user would pinch the spring member 1152 inward and insert at least the protruding portion 1121 between wearer's labia. The user would release the device, allowing the spring member 1152 to spring back toward an original shape or size, leveraging the inside of the labia tissue to secure the fluid collection device 1100 in position. Accordingly, the fluid collection device 1100 would be secured via the labia only. Alternatively or additionally, other securement approaches may be utilized such as adhesive tabs, use positioning in the gluteal cleft, straps around waist and/or buttocks area, or the like.

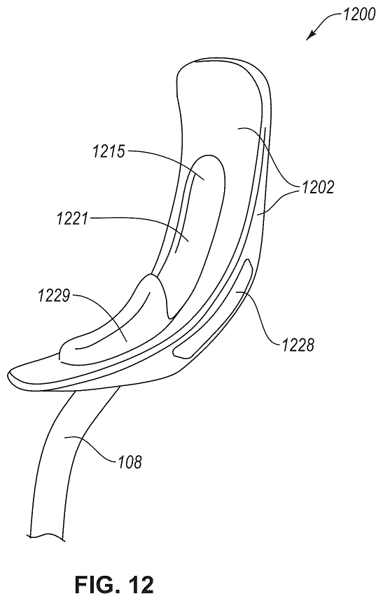

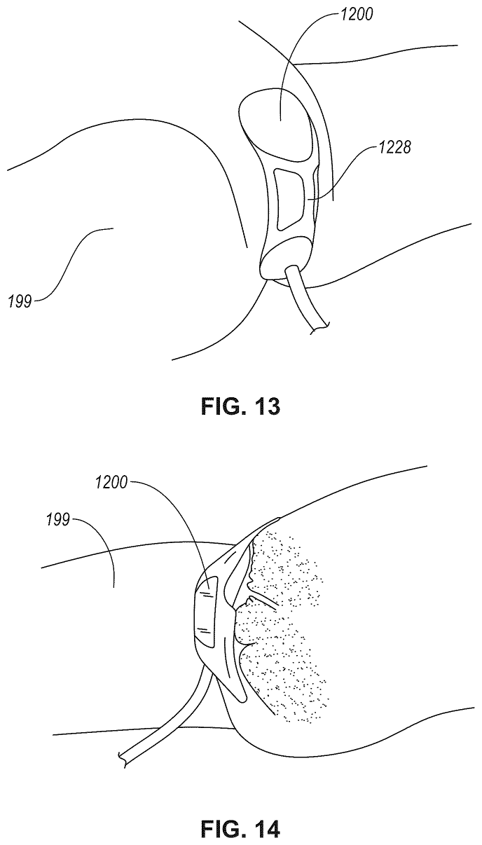

In some examples, the fluid collection devices may include elements to promote gripping by the user (e.g., wearer or health care professional), such as to aid the user to position the device on a wearer. Further, the fluid impermeable barrier may extend over a portion of the protruding portion of the fluid permeable body. FIG. 12 is an isometric view of a fluid collection device 1200, according to an embodiment. FIG. 13 is an isometric view of the fluid collection device 1200 of FIG. 12 positioned on a wearer. FIG. 14 is side view of the fluid collection device 1200 of FIG. 12 positioned on a wearer. The fluid collection device 1200 includes the fluid impermeable barrier 1202, the fluid permeable body 1215, and the conduit 108.

The fluid permeable body 1215 may be similar or identical to any of the fluid permeable bodies disclosed herein, in or more or aspects. For example, the fluid permeable body 1215 may include a fluid permeable membrane and a fluid permeable support as disclosed herein. The fluid permeable body 1215 includes the protruding portion 1221, which extends above at least a portion of the substantially flat arcuate surface. The protruding portion 1221 may be sized and shaped to fit between the labia of a wearer. For example, the protruding portion 1221 may be shaped as a substantially hemi-cylindrical body extending longitudinally along the wearer-facing surface of the fluid permeable body 1215.

The fluid impermeable barrier 1202 may be similar or identical to any of the fluid impermeable barriers disclosed herein, in one or more aspects. The fluid impermeable barrier 1202 exhibit a wearer-facing side having a generally flat arcuate shape sized and shaped to contour the anatomy of a wearer's perineal and pubic regions. The fluid impermeable barrier 1202 may include a covering portion 1229 that extends over some of the protruding portion 1221, such as a lower portion thereof. The covering portion 1229 may contour the protruding portion 1221 and extend above the flat arcuate shape of the fluid impermeable barrier 1202. Accordingly, the opening 1206 is defined at least in part by the covering portion 1229 and the flat arcuate shape on the wearer-facing side of the fluid impermeable barrier 1202. The covering portion 1229 is sized and shaped to fit between the labia of the wearer.

The covering portion 1229 may be disposed over a lower portion of the fluid impermeable barrier 1202, such as to form a sump, gutter, or trough in the fluid impermeable barrier 1202. The covering portion 1229 may be used to facilitate separation of the labia during placement as well as provide a gutter to capture excess urine during use. Additionally, the covering portion 1229 may be sized and shaped to insert into the gluteal cleft for positioning, securement, and retention of the device during use. In such examples, the covering portion 1229 may be positioned between the buttocks of the wearer and the protruding portion 1221 may be positioned between the labia during use.

A back (e.g., opposite the wearer-facing side) of the fluid impermeable barrier 1202 may include a narrow portion 1228 sized and shaped to provide a grip section for gripping or pinching in a hand. The narrow portion 1228 may extend longitudinally along at least a portion of the back of the fluid impermeable barrier 1202.

The trough or sump formed from the covering portion 1229 and the chamber are sized and shaped to accommodate the fluid permeable body 1215 therein. The fluid permeable body 1215 may protrude from the sump to fit between the labia of the wearer (e.g., engage the urethral opening). The sump may be in fluid communication with the conduit via an aperture in the fluid impermeable barrier, such as at a gravimetrically low point therein.

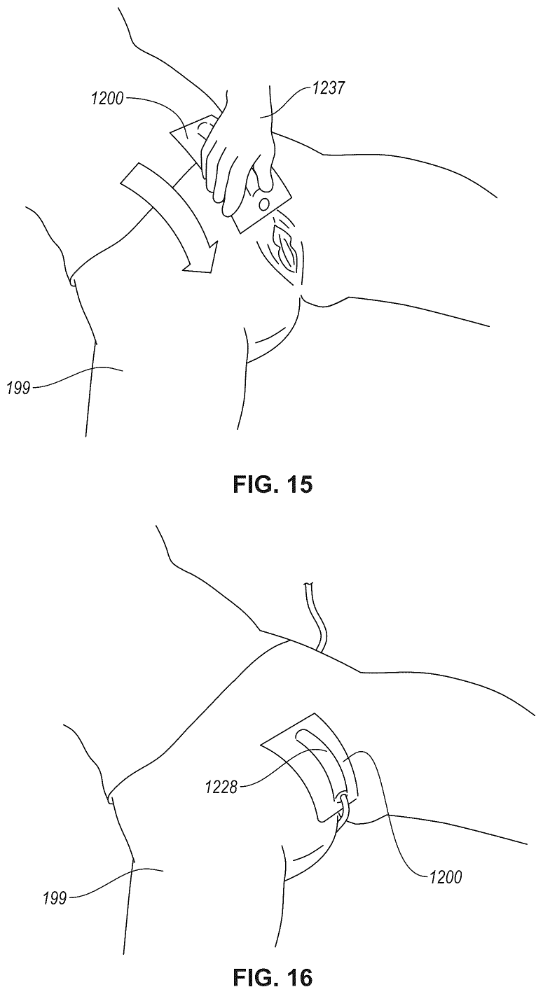

FIGS. 15 and 16 are isometric views of the fluid collection device 1200 of FIG. 12 during and after positioning on a wearer. To position the fluid collection device 1200, a user 1237 would grip the narrow portion 1228 and slide or wiggle the protruding portion 1221 on the opposite side thereof between the labia. For example, the user 1237 may hold the narrow portion 1228 and press the fluid collection device 1200 downward to separate the labia (with the protruding portion 1221). The user 1237 can release the handle when the device is positioned correctly. The wearer 199 (e.g., user or patient) can return to a seated or laying down position.

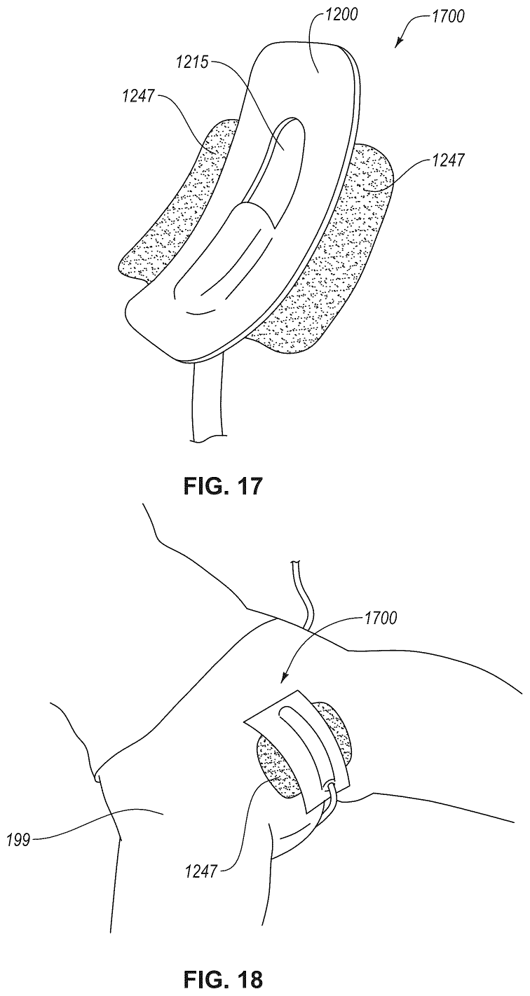

The fluid collection device 1200 may be secured via the labia only or may include additional means for retaining the protruding portion between the labia such as adhesive tabs, straps around waist and/or buttocks area, or any other means disclosed herein. FIG. 17 is an isometric view of a fluid collection device 1700 viewed from the wearer-facing side, according to an embodiment. FIG. 18 is an isometric view of the fluid collection device 1700 disposed on a wearer 199, according to an embodiment. As shown in FIGS. 17 and 18, the fluid collection device 1700 may include the fluid collection device 1200 having adhesive flaps 1247 (e.g., wings) extending therefrom. The adhesive flaps 1247 may be similar or identical to the adhesive flaps 247 in one or more aspects. For example, the adhesive flaps 1247 may be attached and extend from the fluid impermeable barrier of the fluid collection device 1200. The adhesive flaps 1247 includes an adhesive thereon to secure the fluid collection device 1700 to the wearer to maintain the positioning of the protruding portion between the labia. Accordingly, the may adhere to the wearer to maintain the device in a selected position. As shown in FIG. 18, the adhesive flaps 1247 may adhere to the wearer 199 in the groin region. The adhesive flaps 1247 may be sized, positioned, and located to attach to the wearer or the wearer's clothing in the pelvis, buttocks, legs, perineum, or regions thereof. The adhesive flaps 1247 may be an additional means for retaining the protruding portion between the labia of the wearer on the fluid collection device 1200 to form the fluid collection device 1700.

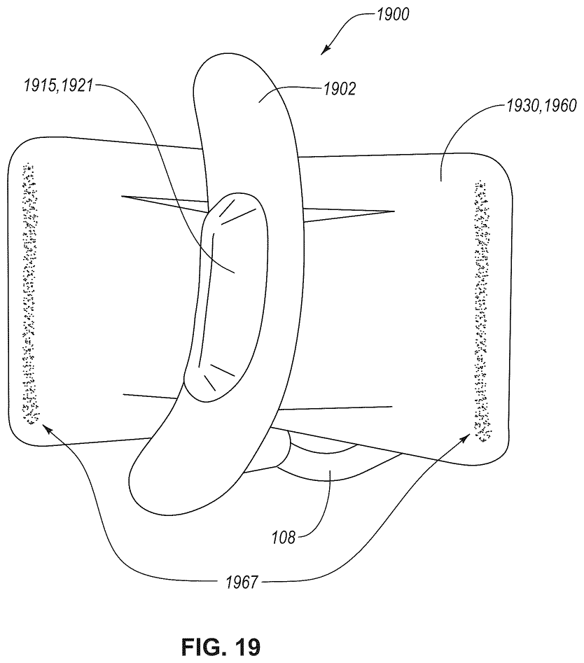

Different styles of flaps, tabs, or means for retaining the protruding portion between the labia of the wearer may be used on a fluid collection device. In some examples, the fluid collection device may include a foldable body sized, shaped, and composed to attach to the fluid impermeable barrier and the wearer to bias the protruding portion toward the wearer during use. FIG. 19 is an isometric view of a fluid collection device 1900, according to an embodiment. The fluid collection device 1900 may be similar or identical to any of the fluid collection devices disclosed herein, in one or more aspects. For example, the fluid collection device 1900 includes the fluid impermeable barrier 1902, the fluid permeable body 1915, the conduit 108, and the means 1930 for retaining the protruding portion between the labia of the wearer. The means 1930 for retaining the protruding portion between the labia of the wearer includes the foldable body 1960. The foldable body 1960 may be attached to the fluid impermeable barrier 1902 and provide a bias (e.g., force) the protruding portion toward the wearer during use.

The fluid impermeable barrier 1902 may be similar or identical to any of the fluid impermeable barriers disclosed herein, on one or more aspects such as materials, shape, or the like. For example, the fluid impermeable barrier 1902 defines a chamber therein, which is in communication with the environment outside of the chamber via an opening in the fluid impermeable barrier 1902. As shown, the fluid impermeable barrier may be substantially cylindrical with the opening extending longitudinally along a longitudinal axis thereof. As shown in FIG. 19, the fluid impermeable barrier 1902 may be tube shaped with a first closed end and a second end having an aperture therein for accommodating the conduit 108 therein. The aperture may extend axially away from the longitudinal axis of the fluid collection device 1900 (to keep the conduit 108 from becoming tangled under or over the wearer during use) or may extend along the longitudinal axis of thereof.

The fluid permeable body 1915 may be similar to any of the fluid permeable bodies disclosed herein in one or more aspects. For example, the fluid permeable body 1915 may include one or more of the fluid permeable membrane, the fluid permeable support, or the protruding portion 1921. The fluid permeable membrane, the fluid permeable support, and the protruding portion 1921, may be similar or identical to fluid any of permeable membranes, the fluid permeable supports, or the protruding portions disclosed herein, in one or more aspects. For example, the protruding portion 1921 is sized and shaped to extend out of and away from the opening of the fluid impermeable barrier 1902 to fit between the labia of the wearer during use. Accordingly, the protruding portion 1921 may be placed between the wearer's labia, such as to contact the urethral opening to promote urine capture. The protruding portion 1921 may receive urine from the urethra of the wearer and the material of the fluid permeable body 115 may facilitate transport of the fluid into the chamber therein for removal therefrom via the conduit 108.

The foldable body 1960 includes a body formed from a foldable material such as a polymer, paper, fabric, or the like. The foldable body 1960 may be at least semi-rigid, such that the non-folded portions of the foldable body 1960 retain at least some rigidity when folded. The foldable body 1960 includes an adhesive 1967 or other attachment means for attaching to the wearer or the wearers clothing. For example, the foldable body 1960 includes adhesive 1967 at the lateral ends thereof. The foldable body 1960 is sized and dimensioned to position the adhesive 1967 at a selected point on the anatomy of the wearer, such as on the inner thighs or groin of the wearer.

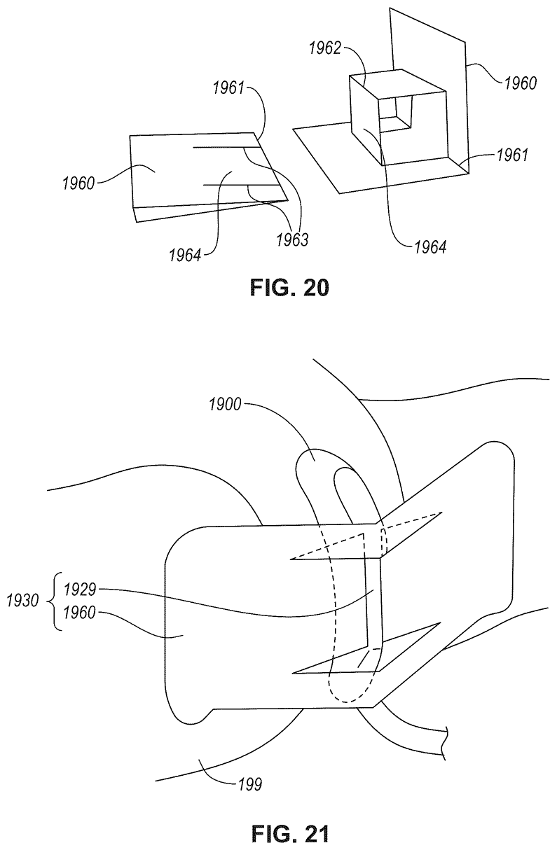

FIG. 20 is an isometric view of the foldable body 1960 in different folded configurations, according to an embodiment. The foldable body 1960 includes a first fold 1961 along a portion (e.g., center) thereof, a cutout portion 1964 defined between cuts 1963 perpendicular to the first fold 1961, and a second fold 1962 in the in the cutout portion 1964, the second fold 1962 being oriented to fold in a direction opposite to the first fold 1961. The cutout portion 1964 is partially separated from the main portion of the foldable body 1960 by the cuts 1963. When opened, the foldable body 1960 is substantially flat. The first and second folds may be substantially collinear when the foldable body 1960 is flat. When bent, the cutout portion 1964 and the second fold 1962 bends away from the first fold 1961. When the first fold 1961 is folded, the second fold 1962 folds in the opposite direction and when the first fold 1961 is unfolded (e.g., body is flattened), the second fold 1962 unfolds (e.g., flattens).

FIG. 21 is an isometric view of the fluid collection device 1900 of FIG. 19 disposed on a wearer 199, according to an embodiment. When the wearer 199 closes their legs, the first fold 1961 bends away from the wearer 199 causing the second fold 1962 to bend toward the labia region. Accordingly, the fluid collection device 1900 attached thereto is pressed against the wearer to ensure securement of the protruding portion between the labia.

The foldable body 1960 may be attached to the fluid collection device 1900 via an adhesive, fasteners, integral formation, or fitting within a portion of the fluid impermeable barrier 1902. In some examples, at least a portion of the fluid impermeable barrier 1902 may be disposed between the first fold 1961 and the second fold 1962. In such examples, the fluid impermeable barrier 1902 may include one or more flanges, cuts, tabs, slots, or other features for retaining a portion the foldable body 1960 thereon or therebeneath. As shown in FIG. 21, the fluid impermeable barrier 1902 may include a flange 1929 longitudinally extending along a back side (e.g., opposite the wearer-facing side and opening) thereof. The flange 1929 may be set-off from at least a portion of the fluid impermeable barrier 1902 to accommodate at least a portion of the foldable body 1960 therebetween. At least a portion of the fluid impermeable barrier 1902 may be disposed between the first fold 1961 and the second fold 1962. Accordingly, the flange 1929 and the foldable body 1960 may make up the means 1930 for retaining the protruding portion between the labia of the wearer. For example, the second fold 1962 of the foldable body 1960 may be disposed beneath the flange 1929 to retain the foldable body 1960 on the fluid impermeable barrier 1902. In some examples, the fluid collection device 1900 may be retained between the labia by any of the other means disclosed herein.

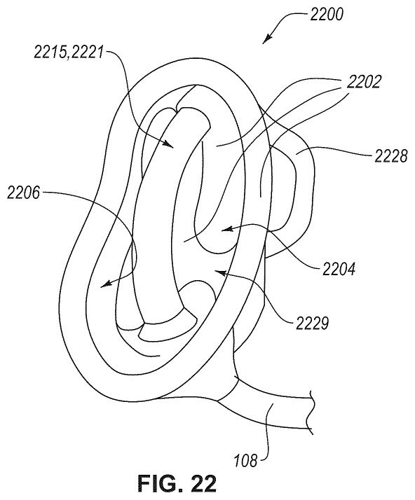

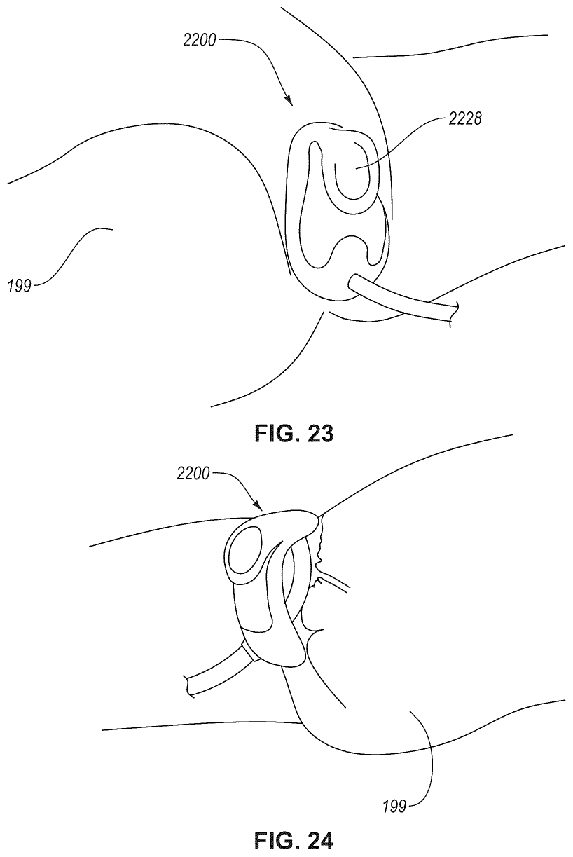

FIG. 22 is an isometric view of a fluid collection device 2200, according to an embodiment. FIGS. 23 and 24 are isometric views of the fluid collection device 2200 of FIG. 22 disposed on a wearer, according to embodiments. The fluid collection device 2200 may be similar or identical to any of the fluid collection devices disclosed herein, in or more aspects. For example, the fluid collection device 2200 includes the fluid impermeable barrier 2202, the fluid permeable body 2215, the conduit 108.

The fluid impermeable barrier 2202 may be similar or identical to any of the fluid impermeable barriers disclosed herein, on one or more aspects such as materials, shape, or the like. For example, the fluid impermeable barrier 2202 defines a chamber 2204 therein, which is in communication with the environment outside of the chamber 2204 via an opening 2206 in the fluid impermeable barrier 2202. The fluid impermeable barrier may be sized and shaped to align with and fit over at least a portion of the labia of the wearer. For example and as shown, the fluid impermeable barrier 2202 may be substantially dome shaped (e.g., convex) with the opening 2206 defined at, and extending around, the lateral base edges thereof (e.g., the mouth of the dome on a wearer-facing side thereof). The outer base defining the opening may be shaped to contour to the anatomy of the vaginal region. The domed shape may be elongated to exhibit a longitudinal axis, a first end region, and a second end region. One or more portions of the fluid impermeable barrier 2202 may be at least semi-rigid to prevent collapse during use.

In some examples, the base of the dome may be at least partially arcuate such as to contour the anatomy of the wearer in the perineal to pubic region along the sagittal plane. As shown in FIG. 24, the base of the dome may be sized and shaped to allow the opening to apply inward pressure (e.g., pinching pressure) from the ends of the arcuate base. Such dimensions may be a means for retaining the protruding portion between the labia of the wearer. In some examples, the fluid impermeable barrier 2202 may exhibit a thicker construction or additional member around the base of the dome, such as to retain shape during use. In some examples, the fluid impermeable barrier 2202 may exhibit a textured surface around the base of the dome, such as to provide a selected amount of grip or tension on the skin of the wearer. The texturing may include a pattern providing a selected amount of surface contact, suction cups, or any other pattern for providing a selected amount of engagement with skin of a wearer. Such texturing may be a means for retaining the protruding portion between the labia of the wearer. The fluid collection device 2200 may be secured to a wearer via an adhesive ring around the fluid impermeable barrier 2202 (e.g., base of dome). Such adhesive may be a means for retaining the protruding portion between the labia of the wearer.

The fluid impermeable barrier 2202 may have a narrow portion 2228 on an outer surface thereof sized and shaped to provide a grip section for gripping or pinching in a hand (e.g., to form a handle). The narrow portion 2228 may protrude from the fluid impermeable barrier 2202. The fluid impermeable barrier 2202 may include one or more transparent portions 2229 for viewing the interior of the device and retraining excess urine therein during use, such as a polymer film. The fluid impermeable barrier 2202 includes an aperture for receiving the conduit 108 in a region thereof expected to be at or near a gravimetrically low point of the fluid collection device during use. For example, the aperture may be located on the first end region of the fluid collection device 2200. The aperture may be in direct communication with the fluid permeable body 2215, such as to allow fluids collected in the fluid permeable body 2215 to be transported to the conduit 108. Such a configuration may prevent fluids from pooling within the dome. The conduit 108 may be in fluid communication with an end of the fluid permeable body 2215. Accordingly, the fluid permeable body 2215 transports urine from wearer's body to the conduit 108, preventing the device from retaining urine.

The fluid permeable body 2215 may be similar to any of the fluid permeable bodies disclosed herein in one or more aspects. For example, the fluid permeable body 2215 may include one or more of the fluid permeable membrane, the fluid permeable support, or the protruding portion 2221. The fluid permeable membrane may be disposed over at least a portion of the fluid permeable support. The fluid permeable membrane or the fluid permeable support may be similar or identical to fluid any of permeable membranes or the fluid permeable supports disclosed herein, in one or more aspects. The fluid permeable body 2215 may be columnar, such as substantially cylindrical, and curved to bend outwardly from the opening 2206. Accordingly, the outward bend of the fluid permeable body 2215 provides the protruding portion 2221. The protruding portion 2221 may be at the apex or apex region of the fluid permeable body 2215. At least the portion of the fluid impermeable barrier 2202 forming the protruding portion 2221 is sized and shaped to fit between the labia of the wearer during use. Accordingly, the protruding portion 2221 may be placed between the wearer's labia, such as to contact the urethral opening to promote urine capture. The protruding portion 2221 may receive urine from the urethra of the wearer and the material of the fluid permeable body 2215 may facilitate transport of the fluid into the conduit 108.

The fluid permeable body 2215 may not be in contact with one or more portions of the interior surface of the fluid impermeable barrier 2202. For example, the fluid permeable body 2215 may be attached to and span across the chamber of the domed fluid impermeable barrier 2202, from one point therein to another point therein. For example, the fluid permeable body 2215 may be attached to the fluid impermeable barrier 2202 near the opening 2206 at the first end region and the second end region to span across the interior region of the chamber 2204. Accordingly, the fluid permeable body 2215 may only contact the fluid impermeable barrier at selected points therein (e.g., in the first and second end regions).

To promote vacuum draw from the wearer and discourage drawing of air, the side of the fluid permeable body 2215 that faces away from the wearer may be covered with a fluid impermeable material, such as a polymer film.

Any of the means for retaining the protruding portion between the labia of the wearer of any of the fluid collection devices disclosed herein may be used with any of the other fluid collection devices disclosed herein, such as in additional to or alternatively to the fluid collection devices described therewith.

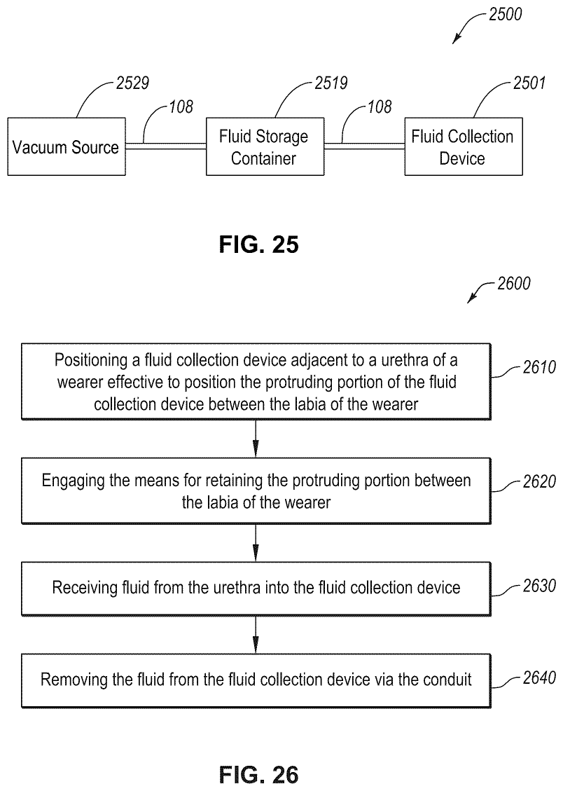

Any of the fluid collection devices disclosed herein may be used in systems for collecting fluids. FIG. 25 is a block diagram of a system 2500 for collecting fluid, according to an embodiment. The system 2500 includes a fluid collection device 2501, a fluid storage container 2519, and a vacuum source 2529. The fluid collection device 2501, the fluid storage container 2519, and the vacuum source 2529 may be fluidly coupled to each other via one or more conduits 108. For example, fluid collection device 2501 may be operably coupled (e.g., fluidly connected) to one or more of the fluid storage container 2519 or the vacuum source 2529 via the conduits 108.

The fluid collection device 2501 may be similar or identical to any of the fluid collection devices disclosed herein. For example, the fluid collection device 2501 may include a fluid impermeable barrier and a fluid permeable body having a protruding portion extending from an opening in the fluid impermeable barrier, as disclosed herein. The fluid collection device May include any of the protruding portions and any combination of the means for retaining the protruding portion between the labia of the wearer disclosed herein. The fluid collection device 2501 may include the conduit 108 including an inlet and an outlet as disclosed herein. The outlet may be fluidly coupled to the fluid storage container 2519 and the inlet may be positioned in the fluid collection device 2501 such as in a portion of the chamber therein selected to be at a gravimetrically low point of the fluid collection device 2501 when worn by a user (e.g., reservoir).

The conduit 108 is coupled to and at least partially extends between one or more of the fluid storage container 2519 and the vacuum source 2529. Accordingly, the vacuum source 2529 may be fluidly connected to the fluid storage container 2519 via the conduit 108. In an example, the conduit 108 is directly connected to the vacuum source 2529. In some examples, the conduit 108 may be indirectly connected to at least one of the fluid storage container 2519 and the vacuum source 2529. In some examples, the conduit 108 may be secured to a wearer's skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety.

Fluid (e.g., urine or other bodily fluids) collected in the fluid collection device 2501 may be removed from the fluid collection device 2501 via the conduit 108. Vacuum or suction force may be applied to remove fluid from the fluid collection device via the conduit either directly or indirectly. The vacuum force may be applied indirectly via the fluid storage container 2519. For example, the second open end of the conduit 108 may be disposed within the fluid storage container 2519 and an additional conduit 108 may extend from the fluid storage container 2519 to the vacuum source 2529. Accordingly, the vacuum source 2529 may indirectly apply vacuum or suction force into the fluid collection device 2501 (e.g., chamber therein) via the fluid storage container 2519 and conduit 108. As the fluid is drained from the chamber of the fluid collection device 2501, the fluid may travel through the first section of conduit 108 to the fluid storage container 2519 where it may be retained. The fluid storage container 2519 is constructed to store fluids therein. The fluid storage container 2519 may be a substantially rigid container, such as a jar, a canister, or the like. The fluid storage container may include a bag in some examples.

The vacuum source 2529 may include one or more of a vacuum pump, a wall-mounted vacuum line, or a hand pump. For example, the vacuum source 2529 may include one or more of a manual vacuum pump, and electric vacuum pump, a diaphragm pump, a centrifugal pump, a displacement pump, a magnetically driven pump, a peristaltic pump, or any pump configured to produce a vacuum. In examples, the vacuum source 2529 may be a portable vacuum source powered by one or more of a power cord (e.g., connected to a power socket), one or more batteries, or even manual power (e.g., a hand operated vacuum pump).

FIG. 26 is a flow diagram of a method 2600 for collecting fluid, according to an embodiment. The method 2600 includes a block 2610 of positioning any of the fluid collection devices disclosed herein adjacent to a urethra of a wearer effective to position the protruding portion between the labia of the wearer; a block 2620 of engaging the means for retaining the protruding portion between the labia of the wearer; a block 2630 of receiving fluid from the urethra into the fluid collection device; and a block 2640 of removing the fluid from the fluid collection device via the conduit. Any the blocks 2610, 2620, 2630, and 2640 of the method 2600 may be performed in different orders, split into multiple acts, modified, supplemented, or combined. For example, the blocks 2610 and 2620 may be combined into a single block. In some examples, one or more of the blocks 2610-2640 of the method 2600 may be omitted.

Block 2610 of positioning any of the fluid collection devices disclosed herein adjacent to a urethra of a wearer effective to position the protruding portion between the labia of the wearer may include utilizing any of the fluid collection devices disclosed herein. Positioning any of the fluid collection devices disclosed herein adjacent to a urethra may include positioning the protruding portion between the labia of the wearer. Positioning any of the fluid collection devices disclosed herein adjacent to a urethra may include positioning the protruding portion in contact with or adjacent to the urethral opening of the wearer.

Block 2620 of engaging the means for retaining the protruding portion between the labia of the wearer includes engaging any of the means for retaining the protruding portion between the labia of the wearer disclosed herein in contact with the wearer or wearer's clothing. In examples, engaging the means for retaining may include adhering or securing the means to the wearer or the wearer's clothing.

In some examples, the means for retaining the protruding portion between the labia of the wearer may include adhesive flaps disposed on the fluid impermeable barrier, and engaging the means for retaining the protruding portion between the labia of the wearer includes adhering the adhesive flaps to the wearer or clothing of the wearer.

In some examples, the means for retaining the protruding portion between the labia of the wearer may include one or more strap sized and configured to wrap around one or more anatomical features of the wearer, and engaging the means for retaining the protruding portion between the labia of the wearer includes wrapping the one or more straps around at least one of the legs, pelvis, or buttocks of the wearer.

In some examples, the means for retaining the protruding portion between the labia of the wearer may include an additional protrusion extending from the first end region of the fluid impermeable barrier on a wearer-facing side thereof, and engaging the means for retaining the protruding portion between the labia of the wearer includes positioning the additional protrusion between the buttocks of the wearer.

In some examples, the fluid impermeable barrier may be shaped as a trough and the opening is disposed at a wearer-facing side of the trough. In such examples, the means for retaining the protruding portion between the labia of the wearer may include a plurality of spring members extending longitudinally along the opening, the plurality of spring members being configured to provide an outward bias on the opening. In such examples, engaging the means for retaining the protruding portion between the labia of the wearer may include pinching the fluid impermeable barrier to bias the plurality of spring members inward, placing the protruding portion between the labia of the wearer, and releasing the plurality of spring members. The spring members will allow the fluid impermeable barrier to at least partially retain its shape and provide some engagement between the labia and the fluid impermeable barrier and the fluid permeable body.

In some examples, the means for retaining the protruding portion between the labia of the wearer may include a foldable body configured to attach to the fluid impermeable barrier and the wearer and to bias the protruding portion toward the wearer. In such examples, engaging the means for retaining the protruding portion between the labia of the wearer includes attaching lateral ends of the foldable body to the inner thighs of the wearer, such as via an adhesive thereon.

In some examples, the fluid impermeable barrier may include a substantially domed shape and sized to at least partially fit over the labia of the wearer and the opening is defined at a base edge of the domed shape, the fluid permeable body is attached to the chamber of the fluid impermeable barrier at the first end region and the second end region, and the fluid permeable body is substantially cylindrical and curved to bend outwardly from the opening. In such examples, positioning any of the fluid collection devices disclosed herein adjacent to a urethra of a wearer effective to position the protruding portion between the labia of the wearer includes positioning the curved fluid permeable body between the labia of the wearer. The means for retaining the protruding portion between the labia of the wearer may include the opening of the fluid impermeable barrier. In such examples, engaging the means for retaining the protruding portion between the labia of the wearer may include contacting the opening of the fluid permeable body to the wearer, such as around at least a portion of the labia of the wearer, effective to adhere or provide frictional engagement with the wearer.

In some examples, the means for retaining the protruding portion between the labia of the wearer may include valleys disposed on the wearer facing surface of the fluid collection device. In such examples, engaging the means for retaining the protruding portion between the labia of the wearer may include disposing the valleys in the wearer facing surface of the fluid collection device over the labia of the wearer, such as to at least partially contain the labia therein.

Engaging the means for retaining the protruding portion between the labia of the wearer may include engaging any combination of means disclosed herein with the wearer or the wearer's clothing.

Block 2630 of receiving fluid from the urethra into the fluid collection device may include receiving the fluid into fluid permeable body, such as via the protruding portion. Receiving fluid from the urethra into the fluid collection device may include wicking the fluid into the chamber of the fluid collection device via the fluid permeably body (e.g., fluid permeable membrane and fluid permeable support). Receiving fluid from the urethra into the fluid collection device may include flowing the fluid towards a portion of the chamber of the fluid collection device that is fluidly coupled to one or more of a gravimetrically low portion of the fluid chamber, an inlet of a conduit, or a reservoir.

Block 2640 of removing the fluid from the fluid collection device via the conduit may include removing at least some of the fluid from the chamber of the fluid collection device. Removing the fluid from the fluid collection device via the conduit may include removing at least some of the fluid from the fluid permeable body of the fluid collection device. Removing the fluid from the fluid collection device via the conduit may include applying vacuum in the chamber, such as via any of the vacuum sources disclosed herein. In such examples, the removing may include activating the vacuum source. Removing the fluid from the fluid collection device via the conduit may include removing the fluid into a fluid storage container.

The method 2600 may include collecting the fluid(s) that are removed from the fluid collection device, such as in the fluid storage container. The method may include one or more of testing or quantifying the amount of fluid removed.

The fluid collection devices, systems, and methods disclosed herein provide for immediate control of fluids emitted from a wearer via the protruding portion of the fluid permeable body as well as secure positioning of protruding portion between the labia of a wearer, which prevents leakage and soiling of the wearer's clothing, bedding, and care takers.

As used herein, the term “about” or “substantially” refers to an allowable variance of the term modified by “about” by ±10% or ±5%. Further, the terms “less than,” “or less,” “greater than”, “more than,” or “or more” include as an endpoint, the value that is modified by the terms “less than,” “or less,” “greater than,” “more than,” or “or more.”

While various aspects and embodiments have been disclosed herein, other aspects and embodiments are contemplated. The various aspects and embodiment disclosed herein are for purposes of illustration and are not intended to be limiting.

What is claimed is:

1. A fluid collection device, comprising:
a fluid impermeable barrier at least partially defining a chamber and an opening, the fluid impermeable barrier including a first end region and a second end region;
a fluid permeable body positioned at least partially within the chamber to extend across at least a portion of the opening and configured to allow fluid to move away from the opening, the fluid permeable body including a protruding portion protruding out of the opening and that is sized and shaped to fit between labia of a wearer; and
a conduit disposed within the chamber for removing fluids therefrom.
